(19) United States
(12) Patent Application Publication
(10) Pub. No.: US 2011/0165255 A1
(43) Pub. Date: Jul. 7, 2011

(54) MALIGNANT TUMOR HEAT THERAPY KIT COMPRISING ANTI-REGULATORY T CELL ANTIBODY AND MAGNETIC FINE PARTICLES AND HEAT THERAPY METHOD THEREOF

(76) Inventors: Takeshi Kobayashi, Aichi (JP); Tomio Morino, Aichi (JP)

(21) Appl. No.: 13/062,033

(22) PCT Filed: Sep. 4, 2008
(86) PCT No.: PCT/JP2008/065983

§ 371 Date: Mar. 3, 2011

(51) Int. Cl. A61K 9/14; A61K 39/395; A61P 35/00

(52) U.S. Cl. 424/491; 424/174.1; 424/498

(57) ABSTRACT

The present invention discloses a heat therapy kit for malignant tumor treatment that comprises a pharmaceutical agent containing anti-regulatory T cell antibody and a pharmaceutical agent containing magnetic fine particles, and a heat therapy method that uses that kit.

### TECHNICAL FIELD

The present invention relates to a malignant tumor heat therapy kit, and more particularly, to a malignant tumor heat therapy kit comprising anti-regulatory T cell antibody and magnetic fine particles, and a heat therapy method that uses the same.

### BACKGROUND ART

Surgical therapy, radiation therapy and chemotherapy using anticancer drugs have mainly been used in the past to treat malignant tumors. Due to considerable advances in the areas of diagnostic technology and therapeutic technology, treatment of malignant tumors is improved.

However, malignant tumors still account for more than 30% of the causes of death even today, and development of new treatment methods for malignant tumors are being sought. Development of effective methods for treating metastatic lesions is also desired. Consequently, development has begun on new treatment methods such as gene therapy, immunotherapy and heat therapy.

Among these new treatment methods for malignant tumors, heat therapy is a treatment method that has long been practiced since the time of ancient Greece, and utilizes the property that malignant tumor cells are more susceptible to heat than normal cells. In Japan, heat therapy consists of heating the entire site where malignant tumor tissue is approved.

A known example of method that uses heat therapy for treatment of malignant tumors consists of administering or accumulating magnetic fine particles within a tumor followed by inducing heat generation by irradiating the magnetic fine particles with an alternating magnetic field heat the tumor selectively. The use of magnetite for the heat generating element within the magnetic fine particles in order to improve the therapeutic effect of selectively heating the tumor, and the use of magnetite cationic liposomes (MCL), in which magnetite is coated with lipids having a positive charge, in order to enhance uptake efficiency of the magnetite into malignant tumor cells, are known (Non-Patent Document 1, Non-Patent Document 2).

In the case of heat therapy for malignant tumors, methods that use MCL for the magnetic fine particles are known to induce antitumor immune cells of the host (Non-Patent Document 3, Non-Patent Document 4). In the case of individual animals having multiple cancerous lesions, it has been reported that systemic antitumor immune activity is induced by carrying out heat therapy on a specific cancerous lesion, thereby reducing the size or causing complete regression of those cancerous lesions on which heat therapy was not carried out (Non-Patent Document 5, Non-Patent Document 6). However, there are many cases in which it is difficult to achieve complete regression for other cancerous lesions on which heat therapy was not carried out in various malignant tumors by carrying out heat therapy using magnetic fine particles alone. Thus, there is a need to develop heat therapy capable of achieving regression of cancerous lesions on which heat therapy has not been carried out by more effective induction of systemic antitumor immunity in various malignant tumors.

Although antitumor immunity was described as being induced in malignant tumor heat therapy using magnetic fine particles as described above, regulatory T cells are thought to be involved in this antitumor immunity system. Since these regulatory T cells have an action that suppresses activation of other T cells such as killer and helper T cells, they are known to suppress the function of the total antitumor immune system of a host (Non-Patent Document 7). Since regulatory T cells are known to express cytotoxic T lymphocyte-associated antigen (CTLA4) and the like, it is believed that the antitumor immunity inherently possessed by a host could work more effectively by blocking the function of these regulatory T cells (Non-Patent Document 8). Efforts are currently being made to develop this anti-regulatory T cell antibody (anti-CTLA4 antibody) for use as an antitumor agent.
[Non-Patent Document 1] Shinkai et al. Jpn. J. Hyperthermic Oncol., 10, 168-177 (1994)
[Non-Patent Document 2] Shinkai et al. Biotech. Appl. Biochem, 21, 125-137 (1994)
[Non-Patent Document 3] Ifuji et al. Cancer Immunol Immunother., 52, 80-88 (2003)
[Non-Patent Document 4] Ifuji et al. Cancer Immunol Immunother., 55, 320-328 (2006)
[Non-Patent Document 5] Yanase et al. Jpn. J. Cancer Res. 89, 775-782 (1998)
[Non-Patent Document 6] Ifuji et al. Jpn. J. Hyperthermic Oncol. 21(3), 139-149 (2005)
[Non-Patent Document 7] Tomoyuki Yamaguchi and Shimon Sakaguchi, Seminars in Cancer Biology, 16, 115-123 (2006)
[Non-Patent Document 8] Meloro et. al, Nat Rev Cancer. 7, 95-106 (2007)

### DISCLOSURE OF THE INVENTION

### Problems to be Solved by the Invention

An object of the present invention is to provide heat therapy capable of achieving regression of cancerous lesions on which heat therapy has not been carried out by enhancing systemic antitumor immunity.

### Means for Solving the Problems

As a result of conducting extensive studies to solve the aforementioned problems, the inventors of the present invention found that an effect on a cancerous lesion on which heat therapy has not been carried out can be dramatically improved by combining the use of an antibody against regulatory T cells (anti-regulatory T cell antibody) with malignant tumor heat therapy, and particularly malignant tumor heat therapy that uses magnetic fine particles, thereby leading to completion of the present invention.

Namely, the present invention relates to a malignant tumor heat therapy agent comprising anti-regulatory T cell antibody and magnetic fine particles, and heat therapy using the same. Moreover, the present invention relates to a heat therapy kit characterized in that the magnetic fine particles are magnetic fine particles coated with lipids having a positive charge.

Moreover, the present invention relates to a heat therapy kit characterized in that the magnetic fine particles are magnetic fine particles coated with lipids to which is bound an antibody that selectively binds to malignant tumor cells.

Moreover, the present invention relates to a heat therapy kit characterized in that the anti-regulatory T cell antibody is anti-CTLA4 antibody.

### Effects of the Invention

The present invention is able to achieve regression of cancerous lesions on which heat therapy has not been carried out by enhancing systemic antitumor immunity by combining the use of anti-regulatory T cell antibody and heat therapy using magnetic fine particles.

### BEST MODE FOR CARRYING OUT THE INVENTION

Although any heat generating element can be used for the heat generation within the magnetic fine particles used in the present invention which generates heat by absorbing electromagnetic waves and is harmless to the human body, the elements which generate heat with a frequency of electromagnetic waves that is not absorbed by the human body are particularly advantageous. Examples of which include substances such as a magnetite or ferrite and ferromagnetic metals such as permalloy. The size of the magnetic fine particles is preferably 5 μm or less and particularly preferably 1 μm or less.

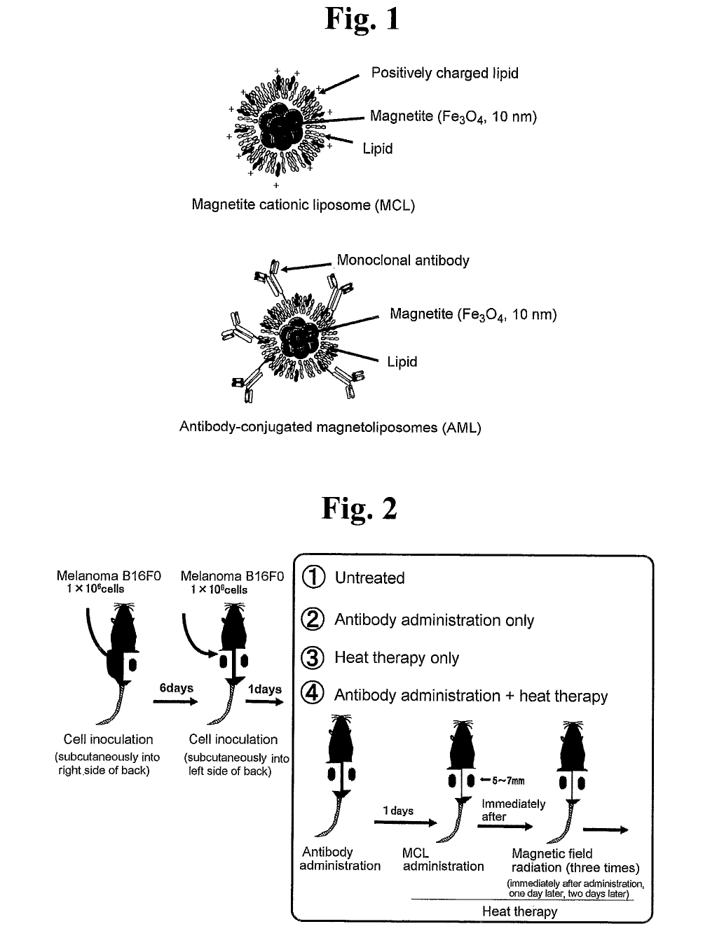

Magnetite cationic liposome (MCL) prepared by coating magnetite with lipids having a positive charge are preferable for use as the magnetic fine particles used in the present invention. The structure of MCL is shown in FIG. 1. Since the surface of malignant tumor cells has a negative charge, cationic MCL bind electrostatically to the malignant tumor cells and are incorporated into the cells by endocytosis. Consequently, MCL injected into malignant tumors are known to remain within the tumors for a long period of time. In addition, magnetic fine particles prepared by conjugating antibody that binds to malignant tumor cells (antibody-conjugated magnetoliposome: AML) can also be used in the present invention. The structure of these AML is shown in FIG. 1. These magnetic fine particles can be produced according to, for example, the method described in Japanese Unexamined Patent Publication No. H3-128331, in which a bifunctional crosslinking agent is bound to magnetic fine particles followed by reacting these magnetic fine particles with antibody that selectively binds to malignant tumor cells.

An alternating magnetic field is preferably used for the magnetic field used in the present invention, and an alternating magnetic field with electromagnetic waves having a frequency of 1 KHz to 10 MHz is particularly preferable.

The anti-regulatory T cell antibody used in the present invention enables to enhance antitumor immunity of a host more effectively by blocking the function of regulatory T cells that suppress activation of other T cells. Although there are no limitations on the kind of anti-regulatory T cell antibody provided it demonstrates the effects of the present invention, examples include anti-CTLA4 antibody and anti-CD25 antibody. In addition, the anti-regulatory T cell antibody used in the present invention includes a fragment thereof that has a function of an anti-regulatory T cell antibody and a protein that contains that fragment, and there are no particular limitations on the production method thereof, and the production method includes known techniques.

All types of malignant tumors are included in the malignant tumor in the present invention. Preferable examples of malignant tumors in the present invention include malignant melanomas and other skin cancer, breast cancer, head and neck cancers, osteosarcoma, lung cancer, colorectal cancer, brain tumors, liver cancer, prostate cancer, pancreatic cancer, renal cancer, esophageal cancer, urinary bladder cancer, ovarian cancer, uterine cancer and gastric cancer, more preferable examples include malignant melanomas and other skin cancer, breast cancer and head and neck cancers, and even more preferable examples include malignant melanomas and other skin cancer.

The present invention relates to the use of an anti-regulatory T cell antibody in malignant tumor heat therapy. Namely, the present invention relates to a malignant tumor heat therapy method comprising administration of an anti-regulatory T cell antibody to an individual having a malignant tumor, and heat therapy for that malignant tumor using magnetic fine particles. More particularly, the present invention relates to malignant tumor heat therapy method comprising administration of an anti-regulatory T cell antibody to an individual with a plurality of malignant tumors, and heat therapy for any of the plural malignant tumors using magnetic fine particles. Administration of anti-regulatory T cell antibody and heat therapy using magnetic fine particles can be carried out simultaneously or at different times and in any order. Although the anti-regulatory T cell antibody is preferably injected intravenously in order to be spread throughout the body, while the magnetic fine particles are preferably injected locally into a malignant tumor, the administration methods thereof are not limited thereto. Moreover, the present invention relates to a malignant tumor heat therapy agent comprising anti-regulatory T cell antibody and magnetic fine particles.

### EXAMPLES

Although the following provides an explanation of the present invention based on examples thereof, the present invention is not limited to these examples.

### Example 1

### Production of MCL

9 mg of dilauroylphosphatidylcholine (Sigma), 9 mg of dioleoylphosphatidyl ethanolamine (Sigma) and 4.5 mg of N-(a-trimethylammonioacetyl)didodecyl-D-glutamate chloride (Sogo Pharmaceutical) were dissolved in 3 ml of chloroform followed by drying under reduced pressure with an evaporator to produce a lipid film on the inner walls of a recovery flask. A 20 mg/2 ml magnetite solution (Toda Kogyo) was added thereto followed by treating with a vortex mixer for about 10 minutes until the lipid film separated from the inner walls of the flask. Ultrasonic treatment for 1 minute and standing over ice for 30 seconds were then repeated until ultrasonic treatment time reached to a total of 1 hour. Then, after adjusting the pH to 7.0 with phosphate buffered saline (PBS), centrifugal separation was carried out for 50 minutes at 10,000 G and 4° C. to recover the MCL as a precipitate followed by suspending in ultrapure water.

### Example 2

### Purification of Regulatory T Cell Antibody

Hybridoma cells of mouse CTLA4 antibody (American Type Culture Collection (ATCC) Number HB-304) were cultured and the culture broth was centrifuged for 10 minutes at 130 G and 4° C. The resulting supernatant again centrifuged for 15 minutes at 10,000 G and 4° C. followed by collection of the supernatant. This supernatant was then cooled with ice and then dissolved by gradually adding 27.7 g of ammonium sulfate per 100 ml while stirring followed by allowing to stand for 1 hour. This was then centrifuged for 5 minutes at 10,000 G and 4° C., and after dissolving the resulting precipitate with about 0.8 to 1.2 ml of sodium phosphate buffer, the solution was dialyzed for 2 hours against the sodium phosphate buffer using a dialysis membrane having a cutoff at a molecular weight of 10,000. Following completion of dialysis, the dialysate was centrifuged for 15 minutes at 10,000 G and 4° C. to obtain a supernatant.

Next, purification was carried out with a Protein G column (HiTrap Protein G HP, GE Healthcare). A sample that had been stored at 4° C. was applied to the column washed with sodium phosphate buffer. After washing with 10 ml of sodium phosphate buffer, 7 ml of glycine-HCl buffer were passed through the column to elute the sample. After adding 200 μl of Tris-HCl buffer to the eluent, the eluent was dialyzed for 2 hours against PBS using a dialysis membrane.

Next, purification was carried out with FPLC (GE Healthcare). The sample was applied using 0.07 M bis-Tris/0.05 M Tris-HCl buffer and eluted with 2 M NaCl solution. Next, the sample was purified with a gel chromatography column (GE Healthcare) using 20 mM sodium phosphate buffer. After measuring the protein concentration of the resulting sample, it was used for animal test as purified monoclonal antibody.

### Example 3

### Animal Testing Model

A model experimental system was produced for the purpose of achieving regression of a cancerous lesion on which heat therapy had not been carried out more effectively by inducing systemic tumor immunity in malignant tumor heat therapy.

Mouse malignant melanoma cells B16F0 (American Type Culture Collection (ATCC) Number CRL-6322) were cultured at 37° C. in the presence of 5% CO₂. After washing the culture dish twice with PBS, the culture dish was treated with trypsin (0.125%) solution. Following treatment, medium was added and the cells were suspended by pipetting followed by centrifuging for 10 minutes at 1,200 rpm and 4° C. to collect the cells. After washing twice with PBS, the cell concentration was adjusted to 1.0×10⁶ cells/100 μl. 100 μl of this cell suspension were transplanted subcutaneously into the right side of the backs of a C57BL/6J mice (Nippon Clea, age 7 weeks, females) using a syringe needle (29 gauge×½″). The same cells were transplanted subcutaneously into the left side of the back using the same procedure at 6 days after inoculation into the right side of the back. The schedule of these tumor transplantation is shown in FIG. 2. Tumor volumes in the mice were calculated using the formula below.

Tumor volume (mm³)=long axis (mm)×short axis (mm)×short axis (mm)/2

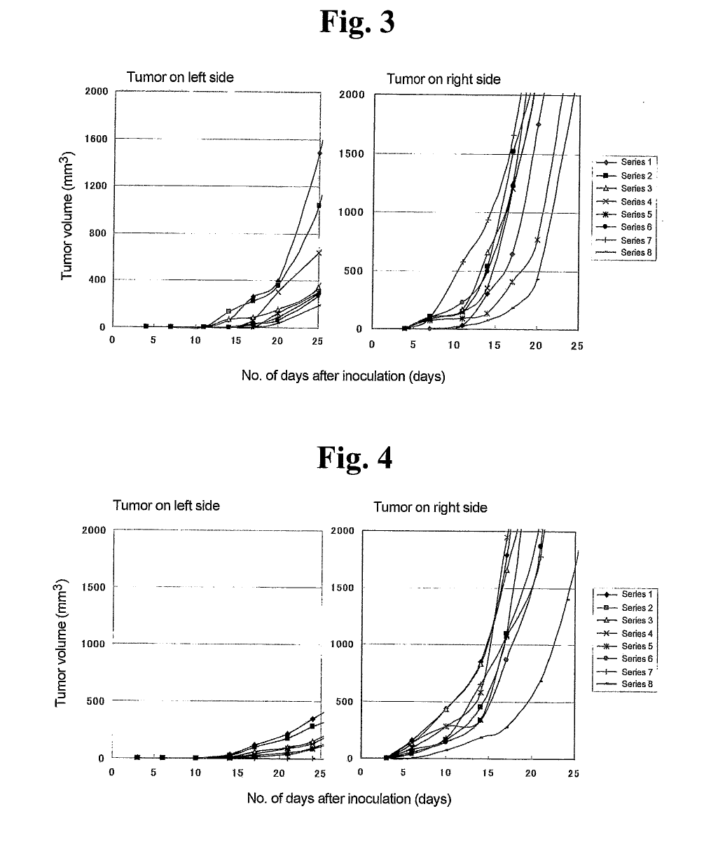

Changes in tumor volume of the left and right sides in this model are shown in FIG. 3. Tumor volumes of the right and left sides were detected at several days after the transplantation of cancer cells, and continued to subsequently become larger (FIG. 3).

### Example 4

### Heat Therapy Method with Administration of Anti-Regulatory T Cell Antibody and Heat Therapy Kit

A heat therapy method was attempted to be developed that is able to achieve regression of cancerous lesions on which heat therapy had not been performed more effectively by enhancing systemic tumor immunity. Namely, antitumor effects against the tumor on the left side of the back in the animal model described in Example 3, on which heat therapy had not been carried out, were examined by carrying out heat therapy on the tumor of the right side. A comparative study was carried out on the following four groups, including treatment combining the use of heat therapy and administration of anti-regulatory T cell antibody: (1) untreated group, (2) antibody dosing group, (3) heat therapy group, and (4) combined group of heat therapy and anti-regulatory T cell antibody dosing.

60 μg of the antibody prepared in Example 2 were administered intraperitoneally at 1 day after the transplantation of tumor cells into the left side of the back (FIG. 2). Heat therapy on the tumor on the right side of the back was carried out at 1 day after the antibody administration. 100 μl of the MCL solution prepared in Example 1 (equivalent to about 3 mg of magnetite) were administered using a syringe pump into the tumor (5 to 7 mm) on the right side of the backs of mice anesthetized with nembutal with 10 to 30 minutes. Following administration, the tumor on the right side of the back was irradiated with an alternating magnetic field of 100 KHz using an alternating magnetic field radiation device (Dai-ichi High Frequency). The surface temperature of the tumor on the right side was measured using a fiber optic thermometer (Model FS600-2M, Anritsu Meter), the output of the radiation device was adjusted so that the temperature reached and kept at 46° C. and, and the radiation was continued on the tumor on the right side for 30 minutes. Magnetic field radiation was carried out in the same manner on the following two days (FIG. 2). Since MCL remains within the tumor, MCL was administered only once prior to the first round of magnetic field radiation. This treatment group was designated as the combined group of heat therapy and anti-regulatory T cell antibody dosing.

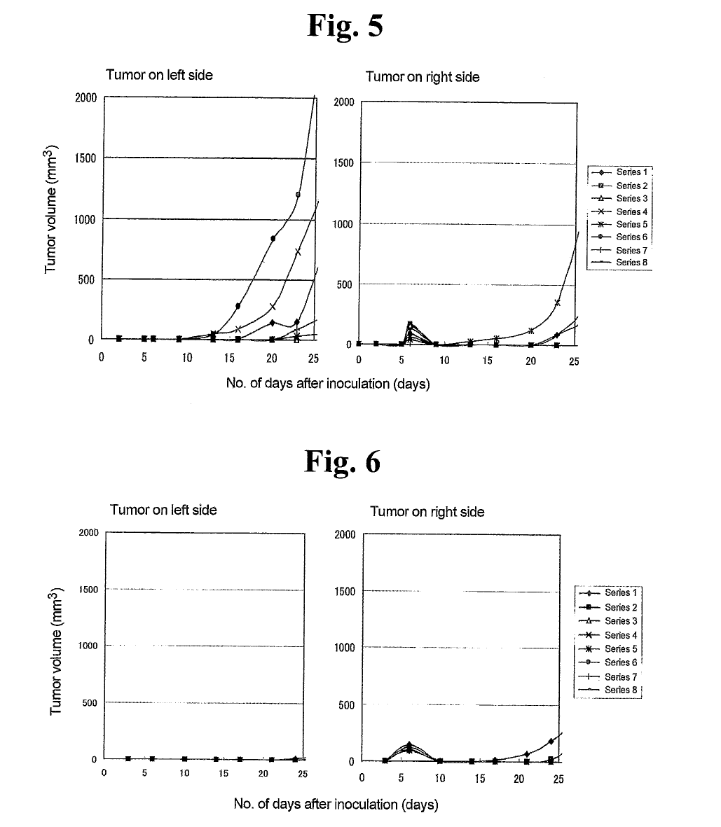

A group that underwent no treatment whatsoever (untreated group), a group that was only administered antibody (antibody dosing group) and a group that only underwent heat therapy (heat therapy group) were also provided as control groups. Changes of tumor volumes on the left and right sides of the back in each group are shown in FIGS. 3, 4, 5 and 6. The untreated group has already been explained in Example 3. In the antibody dosing group (FIG. 4), increases of tumor volume were observed on both the left and right sides. Although growth of the tumor on the left side tended to be slower than the untreated group (FIG. 3), tumor growth was observed in the majority of the animals. Tumor growth on the right side was nearly the same as that of the untreated group. In the heat therapy group (FIG. 5), although regression of the tumor on the right side that underwent heat therapy was observed, growth of the tumors on the left side, which was not subjected to heat therapy, was observed in roughly half of the animals. On the other hand, in the combined treatment group (heat therapy+antibody administration) (FIG. 6), growth of the tumor on the left side that was not subjected to heat therapy was not observed in any of the mice. In addition, prominent tumor regression of the tumor on the right side that was subjected to heat therapy was observed in all animals. On the basis of these findings, combined use of antibody administration and heat therapy for the tumor on the right side was observed to demonstrate antitumor effects on the tumor on the left side that was not subjected to heat therapy. In addition, based on a comparison with the heat therapy group (FIG. 5), combined use with antibody tended to enhance effects on the tumor on the right side that was subjected to heat therapy.

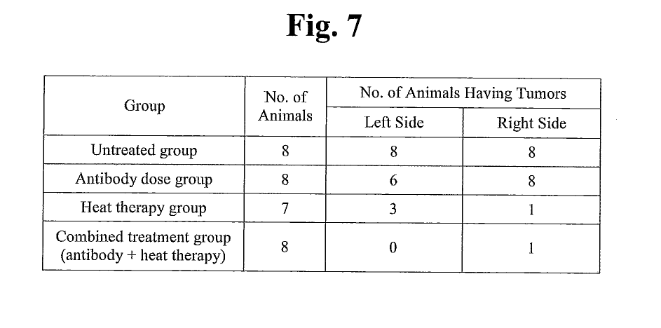

FIG. 7 shows the numbers of animals having tumors on the left or right sides at day 21 after tumor transplant into the right side of the back. The number of tumors on the left side indicates the number of tumors that appeared visually, while the number of tumors on the right side indicates the number of tumors that remained after heat therapy. The presence of a tumor was determined on the basis of palpation and visual examination, the minimum detectable size was about 10 mm³. As shown in FIG. 7, tumors on the left side that were not subjected to heat therapy, were observed in all 8 animals of the untreated group, in 6 of 8 animals in the antibody dosing group, and in 3 of 7 animals of the heat therapy group and in 0 of 8 animals of the combined treatment group (heat therapy+antibody administration).

In this manner, the combined use of heat therapy using magnetic fine particles and administration of anti-regulatory T cell antibody demonstrated that regression can be achieved even in a cancerous lesion on which heat therapy was not carried out, due to enhancing systemic tumor immunity more effectively by using anti-regulatory T cell antibody.

### INDUSTRIAL APPLICABILITY

The present invention of heat therapy agent and treatment method for malignant tumor demonstrate potent antitumor effects even on cancerous lesions at distant sites on which heat therapy is not carried out in comparison with conventional heat therapy agents and heat therapy methods.

FIG. 1 shows the structure of two types of magnetic fine particles used in the present invention, magnetite cationic liposome (MCL) and antibody-conjugated magnetoliposome (AML).

FIG. 2 shows an overall treatment schedule. The schedules for tumor transplantation into the right side of the back, tumor transplantation into the left side of the back, intraperitoneal antibody administration and heat therapy using magnetic fine particles for the tumor on the right side of the back are shown.

FIG. 3 shows changes of tumor volume on the left and right sides in an untreated group.

FIG. 4 shows changes of tumor volume on the left and right sides in an antibody dosing group.

FIG. 5 shows changes of tumor volume on the left and right sides in a heat therapy group.

FIG. 6 shows changes of tumor volume on the left and right sides in a combined group of heat therapy and antibody dosing.

FIG. 7 shows the numbers of animals having tumors on the left or the right side at day 21.

1. A kit for malignant tumor heat therapy, comprising a pharmaceutical preparation containing anti-regulatory T cell antibody and a pharmaceutical preparation containing magnetic fine particles.

2. The kit according to claim 1, wherein the magnetic fine particles are magnetic fine particles coated with lipid having positive charge.

3. The kit according to claim 1, wherein the magnetic fine particles are magnetic fine particles coated with lipid to which is bound an antibody that selectively binds to malignant tumor cells.

4. The kit according to any of claims 1 to 3, wherein the anti-regulatory T cell antibody is anti-CTLA4 antibody.

5. A malignant tumor heat therapy method, comprising administration of anti-regulatory T cell antibody and malignant tumor heat therapy using magnetic fine particles.

6. The heat therapy method according to claim 5, wherein the magnetic fine particles are magnetic fine particles coated with lipid having positive charge.

7. The heat therapy method according to claim 5, wherein the magnetic fine particles are magnetic fine particles coated with lipid to which is bound an antibody that selectively binds to malignant tumor cells.

8. The heat therapy method according to any of claims 5 to 7, wherein the anti-regulatory T cell antibody is anti-CTLA4 antibody.
